(19) United States
(12) Patent Application Publication
(10) Pub. No.: US 2009/0137836 A1
(43) Pub. Date: May 28, 2009

(54) PROCESSES FOR PREPARING LOW-ACID POLYALKYLENE TEREPHTHALATE FROM DIOL-CAPPED PRE-POLYMER AND LINEAR OLIGOMER RECYCLATE AND USING SUCH IN THE PRODUCTION OF MACROCYCLIC POLYESTER OLIGOMER

(76) Inventors: Peter D. Phelps, Williamstown, MA (US); Paul M. Andrusyszyn, New Castle, DE (US)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 12/257,283

(22) Filed: Oct. 23, 2008

Related U.S. Application Data
(60) Provisional application No. 60/981,942, filed on Oct. 23, 2007.

(51) Int. Cl. C07C 69/76

(52) U.S. Cl. 560/89

(57) ABSTRACT

The invention relates to a method for producing low-acid polyalkylene terephthalate from which MPO can be advantageously manufactured. In certain embodiments, low-acid polybutylene terephthalate (PBT) is prepared by reacting low molecular weight, diol-stopped PBT pre-polymer with a linear oligomer recyclate in refluxing ortho-dichlorobenzene (oDCB) solvent. The linear oligomer recyclate may be a by-product of the depolymerization/cyclization of the low-acid PBT, thereby synergistically increasing overall conversion of reactants to the MPO product, cyclic polybutylene terephthalate (cPBT).

### RELATED APPLICATIONS

This application claims benefit of U.S. Provisional Patent Application No. 60/981,942, filed on Oct. 23, 2007, the text of which is incorporated herein by reference in its entirety.

### FIELD OF THE INVENTION

This invention relates generally to the manufacture of low-acid polyalkylene terephthalate (such as polybutylene terephthalate, PBT) for conversion to macrocyclic polyester oligomer (such as cyclic polybutylene terephthalate, cPBT). More particularly, in certain embodiments, the invention relates to manufacture of low-acid polyalkylene terephthalate (such as PBT) from low molecular weight, diol-stopped pre-polymer and linear oligomer recyclate in refluxing solvent (such as ortho-dichlorobenzene, oDCB); the low-acid polyalkylene terephthalate is suitable for advantageous conversion to macrocyclic polyester oligomer (MPO).

### BACKGROUND OF THE INVENTION

International (PCT) Patent Application No. PCT/US2006/010541, (the '541 application) was filed on Mar. 24, 2006, was published as WO2006/104821 on May 10, 2006, and is incorporated herein by reference in its entirety. The '541 application describes systems and methods for preparing low-acid polyalkylene terephthalate (such as polybutylene terephthalate, PBT) from which macrocyclic polyester oligomer (MPO) can be advantageously manufactured. Depolymerization of low-acid polyalkylene terephthalate requires less catalyst and proceeds to equilibrium more quickly than depolymerization of higher-acid polyalkylene terephthalate. The use of less catalyst reduces the amount of residual oligomers formed, thereby reducing separation and filtration processing costs. The residual oligomer filtrate that does form is less gellular and easier to remove from a product stream when low catalyst concentrations are used.

In addition to its use in the production of MPO, the low-acid polyalkylene terephthalate is useful in its own right. For example, the low-acid polyalkylene terephthalate can be stabilized to prevent generation of acids, thereby resulting in reduced corrosion problems when used as a polymer in injection molding or other process applications.

In the '541 application, low-acid PBT is produced by reacting butanediol (BDO) and dimethylterephthalate (DMT) in an organic solvent such as ortho-dichlorobenzene (oDCB) in the presence of a catalyst at about atmospheric pressure and at about the boiling point of the solvent (for example, less than about 200° C.). However, preparation of the low-acid PBT adds to the overall cost of preparing MPO. Even though depolymerization of commercially-available PBT requires more catalyst and may result in an MPO product of lower quality, it may be less expensive to prepare MPO this way, depending on the relative scale of the manufacturing processes involved and the price of the feedstocks used.

There is a need for alternate methods of preparing low-acid polyalkylene terephthalate, such as polybutylene terephthalate (PBT), from which macrocyclic polyester oligomer (MPO), such as cyclic polybutylene terephthalate (cPBT) can be advantageously manufactured.

### SUMMARY OF THE INVENTION

The invention described in the present application relates to methods for producing low-acid polyalkylene terephthalate from which MPO can be advantageously manufactured. Low-acid polyalkylene terephthalate (e.g., PBT) is prepared by reacting low molecular weight, diol-stopped pre-polymer (e.g., PBT pre-polymer) with a linear oligomer recyclate (e.g., a by-product of MPO production), preferably in a refluxing organic solvent, such as ortho-dichlorobenzene (oDCB). The methods are preferably performed below about 240° C., and more preferably below about 200° C., and can be performed without a vacuum. The low-acid PBT is more suitable for MPO production than commercially-available PBT resins.

In certain embodiments, the linear oligomer recyclate is a by-product of the depolymerization/cyclization of the low-acid polyalkylene terephthalate, thereby synergistically increasing overall conversion of reactants to the MPO product. Furthermore, low molecular weight, diol-capped PBT pre-polymer may be prepared and converted to MPO less expensively than commercially-available PBT.

These methods offer a variety of synergies for coupling PBT and cPBT manufacturing processes. For example, a traditional melt PBT facility or unit operation could produce the low viscosity diol-capped pre-polymer at reduced cost, since the long residence times, high temperature, and high vacuum conditions necessary to build molecular weight would not be required. The acid-stopped linears from the cPBT production process can then be combined with the pre-polymer and effectively recycled.

In one aspect, the invention relates to a method for preparing a low-acid polybutylene terephthalate (PBT) product, where the method includes maintaining a mixture at a temperature no greater than about 240° C. and a pressure at least about atmospheric pressure to produce a low-acid PBT product, the mixture at least initially including a diol-rich PBT pre-polymer, a linear oligomer recyclate from depolymerization of PBT, an organic solvent, and a catalyst. The end caps of the diol-rich PBT pre-polymer are preferably greater than 95 mol. % diol, greater than 98 mol. % diol, greater than 99 mol. % diol, or greater than 99.5 mol. % diol. The pre-polymer preferably has a molecular weight no greater than about 40,000 g/mol, no greater than about 35,000 g/mol, no greater than about 30,000 g/mol, no greater than about 25,000 g/mol, no greater than about 20,000 g/mol, or no greater than about 15,000 g/mol. The solvent preferably includes ortho-dichlorobenzene. In certain embodiments, the low-acid PBT product has acid content no greater than about 15 meq/kg, no greater than about 10 meq/kg, no greater than about 8 meq/kg, or no greater than about 7 meq/kg. In certain embodiments, the diol-rich PBT pre-polymer has not undergone solid state polycondensation prior to its introduction to the linear oligomer recyclate (it is not necessary to use high MW, commercially-manufactured PBT having undergone solid state polycondensation). The mixture is preferably maintained under solvent reflux, and the method includes removing water from the refluxing solvent. In certain embodiments, the weight ratio of the linear oligomer recyclate to the diol-rich PBT pre-polymer is at least initially greater than about 10:90, greater than about 15:85, greater than about 20:80, greater than about 25:75, or greater than about 30:70. In certain embodiments, the linear oligomer recyclate is isolated from a depolymerization process by-product prior to reaction with the diol-rich PBT pre-polymer, wherein the recyclate is isolated by exposure of the depolymerization process by-product to a temperature of at least about 200° C. for at least about 5 minutes thereby allowing precipitation of a metal-containing residue, and by filtration of the depolymerization by-product following precipitation of the metal-containing residue.

In another aspect, the invention relates to a method for preparing a macrocyclic polyester oligomer (MPO), the method including the steps of: (a) maintaining a mixture at a temperature no greater than about 240° C. and a pressure at least about atmospheric pressure and maintaining a concentration of polymer solids in the mixture within a first range to produce a low-acid polyalkylene terephthalate product having acid content no greater than about 10 meq/kg, the mixture at least initially including a diol-rich polyalkylene terephthalate pre-polymer, a linear oligomer recyclate from depolymerization of a polyalkylene terephthalate, an organic solvent, and a catalyst; and (b) reducing the concentration of polymer solids in the mixture following step (a) and maintaining the concentration of polymer solids in the mixture within a second range in the presence of heat, thereby depolymerizing the low-acid polyalkylene terephthalate product from step (a) to produce an MPO. The description of elements of the embodiments above can be applied to this aspect of the invention as well. In certain embodiments, step (a) includes maintaining the mixture under solvent reflux and removing water from the refluxing solvent. In certain embodiments, the linear oligomer recyclate in step (a) is a by-product of the depolymerization of step (b). In certain embodiments, the concentration of polymer solids in the mixture is maintained within a range from about 30 wt. % to about 50 wt. % in step (a), then reduced and maintained within a range from about 0.75 wt. % to about 1.5 wt. % in step (b). The solvent preferably includes ortho-dichlorobenzene.

In certain embodiments, the polyalkylene terephthalate product includes butylene terephthalate units and/or ethylene terephthalate units. Step (a) is preferably conducted at a temperature between about 170° C. and about 210° C. In certain embodiments, the mixture in step (b) further includes a catalyst, which may or may not be the same as the catalyst in step (a). In certain embodiments, the mixture in step (b) includes a titanium depolymerization catalyst at a concentration no greater than about 2 mol Ti per 100 mol alkylene terephthalate repeat units. In certain embodiments, the mixture in step (b) includes a titanium depolymerization catalyst at a concentration from about 0.25 to about 1.25 mol Ti per 100 mol alkylene terephthalate repeat units.

In yet another aspect, the invention relates to a continuous or semi-continuous process for preparing a macrocyclic polyester oligomer by depolymerizing low-acid polybutylene terephthalate, the process including: (1) a first unit operation for producing a low-acid polybutylene terephthalate product, wherein the first unit operation maintains a first mixture at a temperature no greater than about 240° C. and a pressure at least about atmospheric pressure, the first mixture at least initially including a diol-rich polybutylene terephthalate pre-polymer, a linear oligomer recyclate from depolymerization of PBT, an organic solvent, and a catalyst, and wherein an output stream including the low-acid PBT product flows from the first unit operation to a second unit operation; and a second unit operation for depolymerization of the low-acid PBT, wherein the second unit operation exposes a second mixture including the low-acid PBT to heat in the presence of a depolymerization catalyst, thereby producing a macrocyclic polyester oligomer. The description of elements of the embodiments above can be applied to this aspect of the invention as well.

In preferred embodiments, the first unit operation includes maintaining the mixture under solvent reflux and, preferably, water is removed from refluxing solvent. In certain embodiments, the concentration of polymer solids in the first mixture is maintained within a range from about 30 wt. % to about 50 wt. %, and the polymer solids concentration of the second mixture is maintained within a range from about 0.75 wt. % to about 1.5 wt. %. The solvent preferably includes ortho-dichlorobenzene.

In certain embodiments, linear oligomer recyclate in the first unit operation is a by-product of the depolymerization of PBT in the second unit operation. In certain embodiments, the low-acid PBT product from the first unit operation has acid content no greater than about 15 meq/kg, no greater than about 10 meq/kg, no greater than about 8 meq/kg, or no greater than about 7 meq/kg. In certain embodiments, the weight ratio of the linear oligomer recyclate to the diol-rich PBT pre-polymer is at least initially greater than about 10:90, greater than about 15:85, greater than about 20:80, greater than about 25:75, or greater than about 30:70.

In certain embodiments, the second mixture includes a titanium depolymerization catalyst at a concentration no more than about 2 mol Ti per 100 mol butylene terephthalate repeat units. In certain embodiments, the second mixture includes a titanium depolymerization catalyst at a concentration from about 0.25 to about 1.25 mol Ti per 100 mol butylene terephthalate repeat units.

In certain embodiments, the process further comprises a third unit operation for isolating the linear oligomer recyclate, wherein the third unit operation exposes an output stream of a depolymerization process to a temperature of at least about 200° C. for at least about 5 minutes, thereby allowing precipitation of a metal-containing residue, and wherein the third unit operation filters the depolymerization process output following precipitation of the metal-containing residue, thereby isolating the linear oligomer recyclate.

The description of the embodiments of one aspect of this invention may be applied to other aspects of the invention as well.

### BRIEF DESCRIPTION OF THE DRAWINGS

The objects and features of the invention can be better understood with reference to the drawings described below, and the claims. The drawings are not necessarily to scale, emphasis instead generally being placed upon illustrating the principles of the invention. In the drawings, like numerals are used to indicate like parts throughout the various views.

FIG. 1 is a process flow diagram depicting unit operations in a process for producing a low-acid polyalkylene terephthalate, according to an illustrative embodiment of the invention.

FIG. 2 is a process flow diagram depicting unit operations in a process for producing a macrocyclic polyester oligomer by polymerizing and subsequently depolymerizing/cyclizing a low-acid polyalkylene terephthalate, according to an illustrative embodiment of the invention.

FIG. 3 is a graph depicting PBT molecular weight as a function of reaction time in a reaction mixture according to an illustrative embodiment of the invention.

### DETAILED DESCRIPTION

It is contemplated that compositions, mixtures, systems, methods, and processes of the claimed invention encompass variations and adaptations developed using information from the embodiments described herein. Adaptation and/or modification of the compositions, mixtures, systems, methods, and processes described herein may be performed by those of ordinary skill in the relevant art.

Throughout the description, where systems are described as having, including, or comprising specific components, or where processes and methods are described as having, including, or comprising specific steps, it is contemplated that, additionally, there are systems of the present invention that consist essentially of, or consist of, the recited components, and that there are processes and methods according to the present invention that consist essentially of, or consist of, the recited processing steps.

Similarly, where mixtures and compositions are described as having, including, or comprising specific compounds and/or materials, it is contemplated that, additionally, there are mixtures and compositions of the present invention that consist essentially of, or consist of, the recited compounds and/or materials.

It should be understood that the order of steps or order for performing certain actions is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

The '541 application describes systems and methods for preparing low-acid polyalkylene terephthalate from which macrocyclic polyester oligomer (MPO) can be advantageously manufactured, for example, using less titanium catalyst. The invention described in the present application introduces an alternate approach for producing low-acid polyalkylene terephthalate from which MPO can be advantageously manufactured. In certain embodiments, the acid content of commercially available polybutylene terephthalate (PBT) is reduced by adding a small amount of 1,4-butane diol (BDO) to a solution of the commercially available PBT in refluxing ortho-dichlorobenzene (oDCB) solvent. The low-acid PBT is then advantageously depolymerized to form cyclic polybutylene terephthalate (cPBT).

In other embodiments, a low-acid PBT is formed by reacting a diol-rich pre-polymer with low molecular weight, acid-stopped linears in refluxing ortho-dicholorobenzene (oDCB) solvent, such linears being a by-product of an MPO production process (e.g., a by-product of the depolymerization of PBT to form cPBT). The low-acid PBT is then advantageously depolymerized to form cPBT.

Low-acid PBT may also be prepared using a combination of the methods presented, e.g., preparation from (i) commercially-available PBT and BDO in oDCB, (ii) reaction of linear oligomer recyclate and diol-rich PBT pre-polymer in oDCB, and/or (iii) reaction of BDO and DMT in oDCB. The low-acid PBT and may then be depolymerized to form cPBT, according to methods described herein.

Low-acid PBT prepared as described herein may also be isolated and stabilized to prevent acid formation, and used as an engineering thermoplastic resin. Such low-acid PBT resins exhibit improved polymer properties, for example, increased hydrolytic and thermal stability, due to the low-acid content.

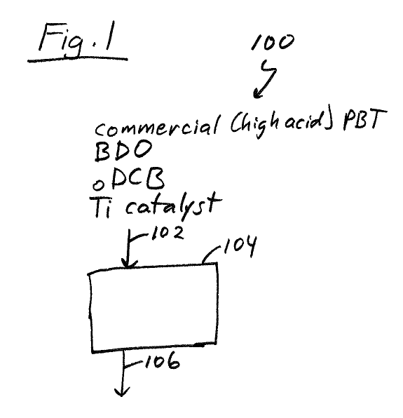

FIG. 1 is a flow diagram 100 depicting a process for producing a low-acid polyalkylene terephthalate, according to an illustrative embodiment of the invention. In the example depicted in FIG. 1, commercially-available (high acid) PBT reacts with a small amount of BDO in oDCB solvent in the presence of a catalyst to produce low acid PBT. One or more input streams 102 provide reactants including commercially-available PBT and BDO in a reactor 104. The one or more input streams 102 also provide solvent (e.g., ortho-dichlorobenzene, oDCB), and a titanium catalyst (e.g., tetraisopropyl titanate, TPT). The reaction mixture is maintained at about the boiling point of the solvent at atmospheric pressure, and water is removed from the refluxing solvent. The output stream 106 may be filtered for removal of non-PBT species, and/or stabilized to prevent formation of acid species. The low-acid PBT filtered from the output stream 106 may be pelletized, shaped, or otherwise processed so that the resulting PBT product is in a form that is convenient for use or transport. Alternatively, the low-acid PBT may be used directly as input in a depolymerization/cyclization process for the advantageous production of cPBT, as described elsewhere herein.

Typical commercial grades of PBT (feedstock PBT) have degree of polymerization from about 80 to about 220. It is desired to use enough BDO to convert substantially all of the acid end groups of the feedstock PBT to alcohols (hydroxybutyl ester end groups). It is found that use of BDO in an amount from about 2.0 g to about 15 g BDO per kg of PBT provides sufficient conversion of acid end groups, and, preferably, from about 3.7 to about 10.3 g BDO per kg of PBT.

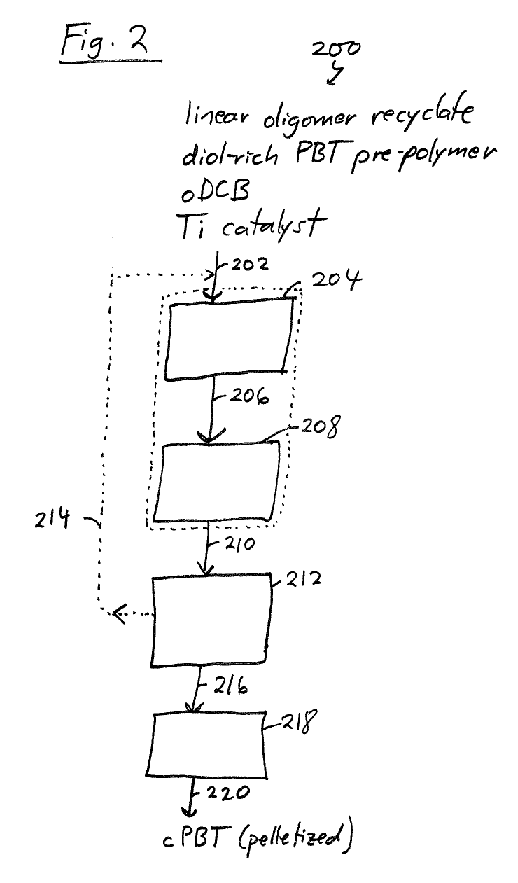

FIG. 2 is a flow diagram 200 depicting a process for producing a macrocyclic polyester oligomer by preparing a low-acid polyalkylene terephthalate (e.g., low-acid PBT) and subsequently depolymerizing/cyclizing the low-acid polyalkylene terephthalate to form MPO (e.g., cPBT). In the example depicted in FIG. 2, linear oligomer recyclate and diol-rich PBT pre-polymer react to produce low-acid PBT, which is depolymerized (cyclized) to form cPBT. The output stream 206 of the reaction step 204 contains low-acid PBT and is used as input in a depolymerization (cyclization) step 208. It may not be necessary to transfer the polymerization output 206 from one vessel to another, because depolymerization may be conducted using one or more of the reaction vessel(s) used in the reaction step 204. In one embodiment, a single unit operation includes both the production of low-acid polyalkylene terephthalate and depolymerization steps. In certain embodiments, the low-acid PBT is, essentially, an intermediate in the production of cPBT. The low-acid PBT produced thusly may be allowed to build to a molecular weight determined to provide improved overall cPBT production rate and/or properties.

The unit operations depicted in the figures may include input and output streams in addition to those shown. For example, in FIG. 2, solvent may be added to dilute the product of the reaction step 204 to levels required for the depolymerization step 208. The process streams shown may contain components other than those listed. The representative contents of process streams are provided for convenience.

In the process of FIG. 2, an output stream 210 of the depolymerization reaction may contain cPBT product in oDCB solvent, as well as byproducts including, for example, residual oligomer, catalyst residue, THF complexes, non-MPO macrocyclic material, and other compounds. The depolymerization output stream 210 may undergo filtration and/or other separation processing 212 so that cPBT product 216 and/or residual oligomers 214 may be extracted. The residual oligomers 214 may be recycled and used as part or all of the linear oligomer recyclate in the input stream 202. The cPBT product 216 can undergo pelletization and/or shaping 218 for conversion into an easily-transportable form 220.

A recyclate stream 214 rich in residual oligomer including, for example, carboxylic acid-terminated linear oligomer species, may be separated from the depolymerization output stream 210. The residual oligomer-rich stream 214 can then be used as input in the reaction step 204, thereby increasing overall conversion of monomers to cPBT. It is preferable, but not required, to remove catalyst residue before using oligomer byproduct as recyclate in the reaction step 204, for example, using methods described in the '541 application, incorporated by reference herein.

### EXPERIMENTAL EXAMPLES

Experimental examples described herein demonstrate the synthesis of low-acid PBT from: (i) commercially-available PBT and BDO in oDCB, and (ii) reaction of linear oligomer recyclate and diol-rich PBT pre-polymer in oDCB. The low-acid PBT formed thereby was then depolymerized to form cPBT.

Experiments 1 and 2 are control experiments in which no BDO was added. In Experiments 3-10, BDO was added to oDCB solutions of commercially-available PBT pellets at a concentration of about 40 wt. % solids. The commercially-available PBT that was used included Valox 315 grade PBT from GE Plastics and 6550 grade PBT from BASF. The Valox resin had a starting acid concentration of 39.2 mmol/kg and the BASF 6650 resin had a starting acid concentration of 23.7 mmol/kg. In each of Experiments 1-10, a 1 L 3-necked round bottom flask equipped with a mechanical stirrer, a heated reflux condenser fitted with a short path distillation head with receiver, and an inert gas inlet was charged with PBT (220 g or 1 mol repeat units), BDO (various amounts ranging from 235-1140 mg or 0.26 to 1.26 mol % of repeat units), anhydrous oDCB solvent (330 g per 220 g PBT to give 40% reactant solids) and 35 mg fresh of TPT catalyst. BDO and oDCB were used without further purification. The reactants were heated to reflux at about 187° C. (at 40% solids the atmospheric boiling point is about 187 C) and the reacting mixture was sampled to determine the effect on molecular weight (Mw) as equilibration of added BDO took place. After the reaction was held at reflux for the desired time, the polymer solution was poured into a jar. Samples of the 40% PBT/oDCB solid were dried in vacuo at 100° C. to yield dried, powdered polymer.

The PBT powder was then depolymerized to form cPBT. A flame dried, 3-necked 250 ml round bottom flask equipped with a mechanical stirrer, a short path distillation head and condenser, and an inert gas inlet was charged with PBT (approximately 7 mmol or 1.54 g dry wt pellets or powder from solution polymerization to nearest 0.1 mg) and anhydrous oDCB (approximately 110 ml or 143 g to nearest 0.1 mg), then submerged into 220 C oil bath. After the PBT dissolved and several mL of solvent distilled over head, to insure dryness of the reaction, 50 ul of freshly prepared catalyst solution was added to provide an initial Ti concentration of 0.7 mol % vs. PBT repeat units. The catalyst solution was Ti(BD:HG) (4:1) at concentration of 1M in Ti and was prepared according to the methods described in the '541 application, incorporated herein by reference. The reaction was then maintained under a positive pressure of dry nitrogen and sampled at 5, 10 and 15 minutes to determine the initial rate of CBT formation. For the indicated experiments, additional catalyst was added at 15 minutes and a final sample of the reaction was taken at 2 hrs to determine extent of CBT formation by HPLC technique.

In Experiment 11, low-acid PBT was prepared by reacting linear oligomer recyclate and diol-rich PBT pre-polymer in oDCB. Low molecular weight, diol-stopped PBT pre-polymer was prepared by the solution polymerization procedure described in the '541 application, incorporated herein by reference (and attached in the Appendix), using 4% excess BDO. DMT and BDO were reacted in oDCB at atmospheric pressure at about the boiling point of oDCB in the presence of TPT to prepare the PBT pre-polymer. The PBT pre-polymer had a molecular weight of 20.2K and had acid concentration less than 1 mmol/kg, such that virtually all end groups were alcohols. The linear oligomer recyclate consisted essentially of titanium-free (filtered) acid-stopped linears, isolated from a byproduct of PBT depolymerization (cPBT production). Filtration at 180° C. was performed using dried linear waste cake from a cPBT production facility, as described in the above-referenced international patent application. The acid-stopped linears contained 238 mmol/kg acid and were virtually free of titanium.

A 1 L 3-necked round bottom flask equipped with a mechanical stirrer, a heated reflux condenser, and an inert gas inlet was charged with solids (75 wt. % diol-capped PBT pre-polymer and 25 wt. % Ti-free linears), anhydrous oDCB solvent (330 g per 220 g solids to give 40% reactant solids), and 35 mg fresh of TPT catalyst (initial Ti concentration of 0.7 mol % vs. PBT repeat units). oDCB was used without further purification. The reactants were heated to reflux at about 187° C. (at 40% solids the atmospheric boiling point is about 187 C). After the reaction was held at reflux for seven hours, the polymer solution was poured into a jar. Samples of the 40% PBT/oDCB solid were dried in vacuo at 100° C. to yield dried, powdered polymer.

The PBT powder was then depolymerized to form cPBT. A flame dried, 3-necked 250 ml round bottom flask equipped with a mechanical stirrer, a short path distillation head and condenser, and an inert gas inlet was charged with PBT (approximately 7 mmol or 1.54 g dry wt pellets or powder from solution polymerization to nearest 0.1 mg) and anhydrous oDCB (approximately 110 ml or 143 g to nearest 0.1 mg), then submerged into 220 C oil bath. After the PBT dissolved and several mL of solvent distilled over head, to insure dryness of the reaction, 50 ul of freshly prepared catalyst solution was added to provide an initial Ti concentration of 0.7 mol % vs. PBT repeat units. The catalyst solution was Ti(BD:HG) (4:1) at concentration of 1M in Ti and was prepared according to the methods described in the above-referenced international patent application. The reaction was then maintained under a positive pressure of dry nitrogen and sampled at 5, 10 and 15 minutes to determine the initial rate of CBT formation. Additional catalyst was added at 15 minutes and a final sample of the reaction was taken at 2 hrs to determine extent of CBT formation by HPLC technique.

Table 1 shows data obtained from certain of Experimental Examples #3-10 demonstrating the reduction of the acid content of commercially-available PBT by addition of BDO to refluxing solution.

TABLE 1
Preparation of Low-acid PBT from Commercially-available PBT

| PBT Type (Expt. #) | BDO/220 g PBT | | Reflux Time | | Initial Acid in PBT | Final Acid | | Mw vs PS |
|---|---|---|---|---|---|---|---|---|
| BASF 6550 (#3) | 470 | mg | 23 | hr | 23.7 mmol/Kg | 8.6 | mmol/Kg | 76.3K |
| BASF 6550 (#5) | 470 | mg | 1 | hr | 23.7 mmol/Kg | 8.6 | mmol/Kg | 55K |
| BASF 6550 (#6) | 940 | mg | 7 | hr | 23.7 mmol/Kg | 6.8 | mmol/Kg | 55.2K |
| Valox 315 (#8) | 235 | mg | 23 | hr | 39.2 mmol/Kg | 28.1 | mmol/Kg | 80.7K |
| Valox 315 (#9) | 940 | mg | 1 | hr | 39.2 mmol/Kg | 11.0 | mmol/Kg | 56.0K |
| Valox 315 (#10) | 1175 | mg | 2 | hr | 39.2 mmol/Kg | 8.4 | mmol/Kg | 56.0K |

Table 1 demonstrates the effect of the reaction time and the amounts of BDO added on the acid content and final molecular weight of the PBT resin. The acid content of commercially-available PBT was significantly reduced by the methods described above (e.g., from 23.7 mmol/kg to less than 10 mmol/Kg, and from 39.2 mmol/Kg to less than 30, 25, 20, 15, or 10 mmol/Kg).

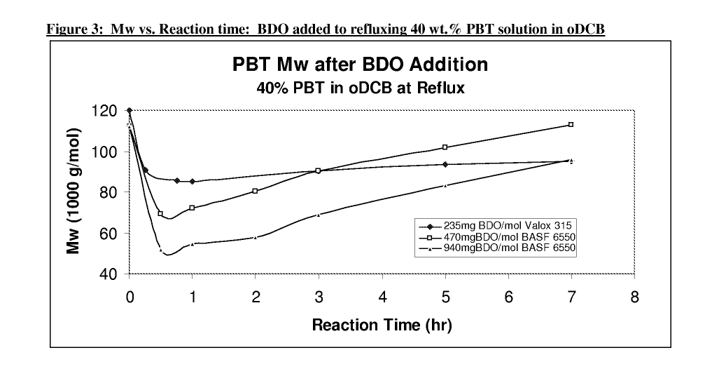

Several of the equilibration reactions were monitored for molecular weight over the course of the reaction. FIG. 3 is a chart showing how molecular weight drop is related to the level of BDO added and how molecular weight recovers over time as end groups react with each other. Using more BDO results in a lower-acid PBT, but causes a steeper molecular weight drop-off. Thus, the amount of BDO and the reaction time can be chosen for the desired acid reduction and molecular weight of the PBT to be depolymerized to form MPO.

Table 2 shows data obtained for Experimental Examples #1-11 during depolymerization of low-acid PBT. The low-acid PBT for experiments #1-11 was prepared by the various methods described above, as denoted in Table 2 by the indicated amounts of initial reactants (e.g., commercial PBT, oDCB, BDO, diol-capped PBT, and/or Ti-free linears) and the indicated solution polycondensation (SP) time (e.g., 1, 2, 7, or 23 hours). Table 2 shows cPBT concentration as a function of time, along with the initial cPBT production rate, and the acid concentration and molecular weight of the PBT after SP, just prior to depolymerization.

TABLE 2
Depolymerization of Low-acid PBT to form cPBT

| Depolymerization to form cPBT | | | cPBT Initial | Acid conc. of | |
|---|---|---|---|---|---|
| (180C Depoly at 0.07M in oDCB | Time | cPBT | Rate | PBT after SP | Mw PBT |
| 0.7% Catalyst) | (hr) | g/L | (g/L/hr) | (mmol/Kg) | (1000 g/mol) |
| Example #1 (control) | | | | | |
| Valox 315 + trace TPT | 0.083 | 0.273 | 0.58 | 39.2 | 71 |
| No added BDO | 0.167 | 0.326 | | | |
| 0.7% then 3% Ti at 15 min | 0.250 | 0.370 | | | |
| | 2.000 | 9.137 | | | |
| Example #2 (control) | | | | | |
| BASF 6550 depoly | 0.083 | 0.558 | 6.17 | 23.7 | 114 |
| No added BDO | 0.167 | 1.117 | | | |
| 0.7% Ti cat at t = 0 | 0.250 | 1.586 | | | |
| no additonal cat at 15 min | 2.000 | 5.559 | | | |
| Example #3 | | | | | |
| 220 g 6550 40% in oDCB | 0.083 | 1.126 | 10.2 | 8.6 | 76.3 |
| 470 mg BDO 23 hr SP | 0.167 | 1.970 | | | |
| 0.7% then 3% Ti at 15 min | 0.250 | 2.823 | | | |
| | 2.000 | 9.207 | | | |
| Example #4 | | | | | |
| 220 g 6550 40% in oDCB | 0.083 | 1.419 | 10.6 | 6.8 | 65.6 |
| 940 mg BDO 7 hr SP | 0.167 | 2.341 | | | |
| 0.7% then 3% Ti at 15 min | 0.250 | 3.192 | | | |
| | 2.000 | 8.799 | | | |
| Example #5 | | | | | |
| 220 g 6550 40% in oDCB | 0.083 | 0.930 | 9.27 | 8.6 | 55 |
| 470 mg BDO 1 hr SP | 0.167 | 1.762 | | | |
| no additonal cat at 15 min | 0.250 | 2.475 | | | |
| | 2.000 | 7.761 | | | |
| Example #6 | | | | | |
| 220 g 6550 40% in oDCB | 0.083 | 1.283 | 11.5 | 6.8 | 55.2 |
| 940 mg BDO 7 hr SP | 0.167 | 2.250 | | | |
| no additonal cat at 15 min | 0.250 | 3.191 | | | |
| | 2.000 | 8.447 | | | |
| Example #7 | | | | | |
| Valox 315 depoly | 0.083 | 0.150 | 1.07 | 39.8 | 120 |
| 0.7% Ti cat at t = 0 | 0.167 | 0.234 | | | |
| no additonal cat at 15 min | 0.250 | 0.329 | | | |
| | 2.00 | 1.460 | | | |
| Example #8 | | | | | |
| 220 g Valox 315 40% in oDCB | 0.083 | 0.208 | 1.48 | 28.1 | 80.7 |
| 235 mg BDO 23 hr SP | 0.167 | 0.292 | | | |
| 0.7% then 3% Ti at 15 min | 0.250 | 0.455 | | | |
| | 2.000 | 9.074 | | | |
| Example #9 | | | | | |
| 220 g Valox 315 40% in oDCB | 0.083 | 0.670 | 3.80 | 11 | 56.0 |
| 940 mg BDO 1 hr SP | 0.167 | 1.048 | | | |
| no additonal cat at 15 min | 0.250 | 1.303 | | | |
| | 2.000 | 3.776 | | | |
| Example #10 | | | | | |
| 220 g Valox 315 40% in oDCB | 0.083 | 1.424 | 9.82 | 8.4 | 56.0 |
| 1175 mg BDO 2 hr SP | 0.167 | 2.349 | | | |
| no additonal cat at 15 min | 0.250 | 3.061 | | | |
| | 2.000 | 8.401 | | | |
| Example #11 | | | | | |
| 75% 20K diol capped | 0.083 | 1.302 | 10.9 | 5.8 | 58.5 |
| 25% Ti Free linears 7 hr SP | 0.167 | 2.290 | | | |
| 0.7% then 3% Ti at 15 min | 0.250 | 3.118 | | | |
| | 2.000 | 8.610 | | | |

Table 2 shows that the acid level in the starting PBT was significantly reduced by both the addition of BDO to commercially-available PBT in refluxing oDCB solvent (Example #3-10), as well as by the reaction of diol-capped PBT pre-polymer with Ti-free linear recyclate in refluxing oDCB solvent (Example #11). Table 2 also shows favorable rates of cPBT formation and ultimate cPBT conversion using only 0.7 mol % Ti catalyst when a low-acid PBT is used. In some cases, the initial rate of conversion to cPBT is significantly increased using the low-acid PBT versus commercially-available PBT (e.g., rates of conversion of at least about 7, 8, 9, 10, or 11 g/L/hr), leading to more efficient, less costly MPO production. Table 2 also shows that it is possible to forego use of additional catalyst (e.g., after 15 minutes of depolymerization), and still obtain high ultimate cPBT conversion rates (e.g., see Example #6). Other processing advantages of using a low-acid PBT for depolymerization to cPBT are as described in the above-referenced '541 application.

### EQUIVALENTS

While the invention has been particularly shown and described with reference to specific preferred embodiments, it should be understood by those skilled in the art that various changes in form and detail may be made therein without departing from the spirit and scope of the invention as defined by the appended claims.

What is claimed is:

1. A method for preparing a low-acid polybutylene terephthalate product, the method comprising maintaining a mixture at a temperature no greater than about 240° C. and a pressure at least about atmospheric pressure to produce a low-acid polybutylene terephthalate product, the mixture at least initially comprising:
(i) a diol-rich polybutylene terephthalate pre-polymer;
(ii) a linear oligomer recyclate from depolymerization of polybutylene terephthalate;
(iii) an organic solvent; and
(iv) a catalyst.

2. The method of claim 1, comprising maintaining the mixture under solvent reflux and removing water from the refluxing solvent.

3. The method of claim 1, wherein the solvent comprises ortho-dichlorobenzene.

4. The method of claim 1, wherein the low-acid polybutylene terephthalate product has acid content no greater than about 10 meq/kg.

5. The method of claim 1, wherein the diol-rich polybutylene terephthalate pre-polymer has not undergone solid state polycondensation prior to introduction to the linear oligomer recyclate.

6. The method of claim 1, wherein the diol-rich polybutylene terephthalate pre-polymer has molecular weight below about 40,000 g/mol.

7. The method of claim 1, wherein the weight ratio of the linear oligomer recyclate to the diol-rich polybutylene terephthalate pre-polymer is at least initially greater than about 10:90.

8. The method of claim 1, wherein the weight ratio of the linear oligomer recyclate to the diol-rich polybutylene terephthalate pre-polymer is at least initially greater than about 25:75.

9. The method of claim 1, wherein the linear oligomer recyclate is isolated from a depolymerization process output prior to reaction with the diol-rich polybutylene terephthalate pre-polymer, wherein the recyclate is isolated by exposure of the depolymerization process output to a temperature of at least about 200° C. for at least about 5 minutes thereby allowing precipitation of a metal-containing residue, and by filtration of the depolymerization process output following precipitation of the metal-containing residue.

10. A method for preparing a macrocyclic polyester oligomer, the method comprising the steps of:
(a) maintaining a mixture at a temperature no greater than about 240° C. and a pressure at least about atmospheric pressure and maintaining a concentration of polymer solids in the mixture within a first range to produce a low-acid polyalkylene terephthalate product having acid content no greater than about 10 meq/kg, the mixture at least initially comprising:
(i) a diol-rich polyalkylene terephthalate pre-polymer;
(ii) a linear oligomer recyclate from depolymerization of a polyalkylene terephthalate;
(iii) an organic solvent; and
(iv) a catalyst; and
(b) reducing the concentration of polymer solids in the mixture following step (a) and maintaining the concentration of polymer solids in the mixture within a second range in the presence of heat, thereby depolymerizing the low-acid polyalkylene terephthalate product from step (a) to produce a macrocyclic polyester oligomer.

11. The method of claim 10, wherein step (a) comprises maintaining the mixture under solvent reflux and removing water from the refluxing solvent.

12. The method of claim 10, wherein the linear oligomer recyclate in step (a) is a by-product of the depolymerization of step (b).

13. The method of claim 10, wherein step (a) comprises maintaining a concentration of polymer solids in the mixture within a first range from about 30 wt. % to about 50 wt. %, and step (b) comprises maintaining a concentration of polymer solids in the mixture within a range from about 0.75 wt. % to about 1.5 wt. %.

14. The method of claim 10, wherein the solvent comprises ortho-dichlorobenzene.

15. The method of claim 10, wherein the polyalkylene terephthalate product comprises butylene terephthalate units and/or ethylene terephthalate units.

16. The method of claim 10, wherein step (a) is conducted at a temperature between about 170° C. and about 210° C.

17. The method of claim 10, wherein the mixture in step (b) comprises a titanium depolymerization catalyst at a concentration no greater than about 2 mol Ti per 100 mol alkylene terephthalate repeat units.

18. The method of claim 10, wherein the mixture in step (b) comprises a titanium depolymerization catalyst at a concentration from about 0.25 mol Ti per 100 mol alkylene terephthalate repeat units to about 1.25 mol Ti per 100 mol alkylene terephthalate repeat units.

19. A continuous or semi-continuous process for preparing a macrocyclic polyester oligomer by depolymerizing low-acid polybutylene terephthalate, the process comprising:
a first unit operation for producing a low-acid polybutylene terephthalate product, wherein the first unit operation maintains a first mixture at a temperature no greater than about 240° C. and a pressure at least about atmospheric pressure, the first mixture at least initially comprising a diol-rich polybutylene terephthalate pre-polymer, a linear oligomer recyclate from depolymerization of polybutylene terephthalate, an organic solvent, and a catalyst, and wherein an output stream comprising the low-acid polybutylene terephthalate product flows from the first unit operation to a second unit operation; and
a second unit operation for depolymerization of the low-acid polybutylene terephthalate, wherein the second unit operation exposes a second mixture comprising the low-acid polybutylene terephthalate to heat in the presence of a depolymerization catalyst, thereby producing a macrocyclic polyester oligomer.

20. The process of claim 19, wherein the first unit operation comprises maintaining the mixture under solvent reflux.

21. The process of claim 19, wherein the linear oligomer recyclate in the first unit operation is a by-product of the depolymerization of polybutylene terephthalate in the second unit operation.

22. The process of claim 19, wherein the low-acid polybutylene terephthalate product from the first unit operation has acid content no greater than about 10 meq/kg.

23. The process of claim 19, wherein the weight ratio of the linear oligomer recyclate to the diol-rich polybutylene terephthalate pre-polymer is at least initially greater than about 10:90.

24. The process of claim 19, further comprising a third unit operation for isolating the linear oligomer recyclate, wherein the third unit operation exposes an output stream of a depolymerization process to a temperature of at least about 200° C. for at least about 5 minutes thereby allowing precipitation of a metal-containing residue, and wherein the third unit operation filters the depolymerization process output following precipitation of the metal containing residue, thereby isolating the linear oligomer recyclate.

25. The process of claim 19, wherein the polymer solids concentration of the first mixture is maintained within a range from about 30 wt. % to about 50 wt. %, and wherein the polymer solids concentration of the second mixture is maintained within a second range from about 0.75 wt. % to about 1.5 wt. %.

26. The process of claim 19, wherein the solvent comprises ortho-dichlorobenzene.

27. The process of claim 19, wherein the second mixture comprises a titanium depolymerization catalyst at a concentration no greater than about 2 mol Ti per 100 mol butylene terephthalate repeat units.

28. The process of claim 19, wherein the second mixture comprises a titanium depolymerization catalyst at a concentration from about 0.25 mol Ti per 100 mol butylene terephthalate repeat units to about 1.25 mol Ti per 100 mol butylene terephthalate repeat units.

29. The process of claim 19, wherein water is removed from refluxing solvent in the first unit operation.
